(19) United States
(12) Patent Application Publication
(10) Pub. No.: US 2010/0047119 A1
(43) Pub. Date: Feb. 25, 2010

(54) DEODORIZER/DISINFECTANT FLUID TREATMENT SYSTEM AND LINER FOR USE THEREWITH.

(76) Inventor: Laura Sutis Cressy, Mandeville, LA (US)

Correspondence Address: JOSEPH T. REGARD, LTD PLC, PO DRAWER 429, MADISONVILLE, LA, 70447-0429, US

(21) Appl. No.: 12/550,546

(22) Filed: Aug. 31, 2009

Related U.S. Application Data
(63) Continuation-in-part of application No. 12/206,441, filed on Sep. 8, 2008, now Pat. No. 7,594,480.
Application No. 12/206,441 is a continuation of application No. 11/094,020, filed on Mar. 30, 2005, now abandoned.

(51) Int. Cl. A61L 2/20; A01K 29/00; A61L 9/00; A61L 9/015

(52) U.S. Cl. 422/5; 119/167; 8/149.2

(57) ABSTRACT

A system for treating and deodorizing objects such as clothing, bedding, or the like, comprising a bag for receiving said objects, and a system for dispersing a deodorizing treatment fluid such as ozone or the like therethrough. Another embodiment of the present invention contemplates a liner to contain and direct a treatment fluid into an absorbent such as pet litter or the like, to disinfect and renew. A complimentary system to the present invention teaches a deodorization, disinfectant, treatment bin for treating litter infused with animal waste, which treatment bin includes a treatment area utilizing ozone to contain and treat the contaminated litter, rendering same re-useable. The system of the present invention further contemplates an ozone generator having a timer circuit for providing a flow of ozone to the apparatus of the present invention, the ozone generator further contemplating as exemplary features a timer apparatus for providing optimal treatment, as well as a proximity sensor system for sensing the presence of an animal in the litter area, for controlling the ozone generator.

### PRIORITY CLAIM

The present application is a continuation in part of U.S. patent application Ser. No. 12/206,441 filed Sep. 8, 2008 listing as inventor Laura Sutis Cressy, and entitled “Deodorizer/Disinfectant Fluid Treatment System and Litter Liner for use Therewith”, which is a continuation of U.S. patent application Ser. No. 11/094,020 filed Mar. 30, 2005 listing as inventor Laura Sutis Cressy, and entitled “Deodorizer/Disinfectant Fluid Treatment System and Litter Liner for use Therewith”.

### TECHNICAL FIELD OF THE INVENTION

The present invention relates to systems for diffusing a fluid deodorizing and/or disinfecting and treatment medium such as ozone gas or the like, the present invention incorporating a fluid conduit associated with a liner for lining an area to be treated.

An embodiment of the present invention teaches a liner for garments or the like hanging on a hanger, and more particularly to a system for lining the garments, the liner having associated therewith a system for dispersing a deodorizing fluid such as ozone gas or the like, which permeates the garment so as to deodorize, freshen, and renew same.

Another embodiment of the present invention teaches a liner for use with such enclosures such as animal litter boxes or the like, and more particularly to a system for lining the container containing waste with an absorption medium, utilizing said liner configured to dispense a treatment fluid throughout said absorption medium, to deodorize and renew same.

A complimentary system to the present invention teaches a deodorization, disinfectant, treatment bin for treating litter infused with animal waste, which treatment bin includes a treatment area which may include a diffusion panel associated therewith for diffusing treatment fluid such as ozone into the treatment area to treat the contaminated litter, rendering same re-useable.

The system of the present invention further contemplates an ozone generator having a timer circuit for providing a flow of ozone to the apparatus of the present invention for a predetermined period, the ozone generator further contemplating as exemplary features a proximity sensor system for sensing the presence of an animal in the litter area, to control the initiation of the treatment process.

### BACKGROUND OF THE INVENTION

The use of deodorizing gas for renewing, refreshing, and deodorizing has been practiced for many years. However, the utilization of such treatment in an effective system for garments and bedding has, it is believed, not been shown. Traditionally, clothing, bedding, or the like, is washed or dry cleaned after it has been worn, even when the items are not visibly dirty, as the clothing or dry cleaning not only cleans the garments but also freshens and deodorizing. Washing and dry cleaning for merely freshening and deodorizing purposes, however, can be expensive and time consuming, not to mention undesirable from an ecological standpoint.

In another application, certain domesticated animals within a household setting, such as cats, are not easily house trained, and require the use of a means of collecting and disposing of liquid and solid waste. A long known and used method of such waste collection and isolation involves the placement of a tray or other container filled with an odor and liquid absorbing medium such as kaolin clay or the like, upon which the animal deposits the waste and typically covers same, allowing the medium to absorb the waste and isolate the odors.

After a specified period of time, the medium becomes infused with the waste and is no longer effective at absorbing and isolating the odors of same. The infused medium must then be disposed, an unsanitary and time consuming process. Further, the cost for renewing the medium can add up over time.

Cat box liners are not particularly new and generally take the form of a bag-like structure whose mouth is folded over the periphery of the tray and the litter or other medium poured thereupon. After the medium is spent, the liner may be removed from the edges of the tray, and lifted to contain the waste infused medium for disposal.

The known prior art has failed to contemplate a system for renewing the waste infused litter situated within the litter box, to effectively extend the time the litter may be used, or further disinfecting the litter box area, or effectively recycle waste infused litter and make same suitable for re-use.

### GENERAL SUMMARY DISCUSSION OF THE INVENTION

The present invention relates to gas and liquid contact apparatus and systems therefore, and particularly to systems for deodorizing, disinfecting, and treating matter, the present invention more particularly contemplating a system utilizing a bag-like liner structure to contain a fluid disinfecting and treatment medium such as ozone or the like, so as to treat clothing, pillows, or bedspreads when they are sealed therein.

A conduit/diffusion structure associated with the interior wall of the liner is formed to convey disinfectant fluid such as ozone or the like into the bag. Ozone or other deodorizing fluid is configured to safely disburse throughout the liner and treat the enclosed items for a specific timed period. When the liner is opened the deodorizing gas will safely disburse into the atmosphere the moment the liner is opened. This system is used to refresh, and disinfect the garments or products made of natural or man-made fabrics. An aerosol dispenser of essential oils may also be utilized in conjunction with the present system, which is sprayed upon or otherwise applied to the clothing or other objects being disinfected, furthering the freshening and deodorizing process.

An alternative embodiment of the present invention contemplates and ozone (or other deodorizing gas) generating structure provided as a component of the bag itself, which structure communicates with disbursing tubes formed on the garment bag, which tubes are formed to disburse the deodorizing gas. The portable ozone generator may be provided into the top of the liner and have a timer, or may be exterior the liner, shown communicating with a conduit formed in the liner via a hose or the like.

The disinfecting and refreshing process is completed in a matter of minutes. A choice of essential oils are part of the refreshing process, but not necessary for the completion of the disinfecting and refreshing. When the bag is opened the ozone is released safely into the atmosphere along with the essential oil fragrance. The liner is designed to fit any travel bag, or used independently. The system may include its own hanger for convenience.

In yet another embodiment of the invention, there is provided a system utilizing a garment/clothing hanger having formed therein a conduit/fluid dispersing system for conveying deodorizing fluid such as ozone gas into a garment bag. The hanger in this embodiment can have a portable deodorizer fluid generator or dispenser (for example, and ozone generator) forming part of the structure of the hanger, or can be adapted to receive a hose or conduit from an exterior generator or dispenser unit.

The present system thereby provides a garment bag formed to refreshen clothing which is well suited for traveling, allowing the user to reduce substantially the amount of washing/dry cleaning required to maintain the clothing/garments in wearable condition.

In Another embodiment of the present invention, there is provided a system used to line a container for containing liquid and/or waste in an absorption medium, utilizing a liner configured which may include a conduit to disperse a deodorizing fluid into the waste, the liner containing the deodorizing fluid about the waste so that the waste is treated by the deodorizing fluid.

The preferred embodiment of the present invention contemplates a liner configured for utilization in conjunction with a cat box containing litter or the like, the liner being formed of thin film or the like and which may have associated therewith a fluid conduit which may or may not contain a further dispersal medium such as a passage, panel, hose or the like to provide a distributed flow of a fluid medium containing, for example, ozone gas or the like. The ozone may also be simply piped into the litter via a hose or other conduit, which may or may not be connected to the liner.

In use, the preferred embodiment of the liner is placed into a cat box or other container to line same, the dispersal and/or diffusion medium preferably associated with the floor and/or lower sidewalls of liner. A litter medium such as kaolin clay or other litter is applied upon the floor of the liner to partially fill the tray. Ozone or another fluid medium is piped to the dispersal or diffusion medium as needed to infuse the litter, and treat any waste associated therewith.

The present system includes apparatus and method for deodorizing an area utilizing a liner associated which may include a diffusion medium, which apparatus and method may be utilized with a variety of applications, and is not limited to the exemplary system associated with a cat box.

A complimentary system to the present invention teaches a deodorization, disinfectant, treatment bin for treating litter infused with animal waste, which treatment bin includes a treatment area having diffusion panel associated therewith for dispensing treatment fluid such as ozone into the treatment area to treat the contaminated litter, rendering same re-useable.

The system of the present invention further contemplates an ozone generator having a timer circuit for providing a flow of ozone to the apparatus of the present invention for a predetermined time, ideally providing sufficient flow of the treatment fluid to fill the litter tray, so as to infuse the litter with treatment fluid, the ozone generator further contemplating as features a delay timer apparatus for providing optimal treatment, as well as a proximity sensor system for sensing the presence of an animal in the litter area, for controlling the ozone generator.

The present system includes apparatus and method for deodorizing an area utilizing a liner associated with a diffusion medium, which apparatus and method may be utilized with a variety of applications, and is not limited to the exemplary system associated with a cat box.

### BRIEF DESCRIPTION OF DRAWINGS

For a further understanding of the nature and objects of the present invention, reference should be had to the following detailed description, taken in conjunction with the accompanying drawings, in which like parts are given like reference numerals, and wherein:

FIG. 1 is an isometric view of the system of the present invention installed in a cat litter box, illustrating a cat approaching the litter box and an ozone generator unit sensing the cat via a proximity sensor, thereby engaging a timing circuit for processing of the cat litter.

FIG. 2 is a side, partial cut-away view of the line or FIG. 1, illustrating a view of the flow conduit formed in the liner for directing the ozonated air to the area of the liner under the cat litter, where it is dispersed/diffused into the cat litter.

FIG. 3 is an isometric view of an exemplary liner of FIG. 1 and its placement into a litter box such that the sides fold over the side walls of the litter box, providing handles for conveying the litter/waste mixture upon completion of use.

FIG. 4 is an isometric view of an exemplary liner and ozone generator of FIG. 1, wherein the dispersal means associated with the liner utilizes a porous, spirally arranged tube which may be affixed to the liner.

FIG. 4A is an isometric view of an embodiment of the present invention, wherein there is provided a hose for dispersing ozone into the tray, which hose may be associated with the liner.

FIG. 5 is an alternative embodiment of the liner of FIG. 4, illustrating a thin film passageway formed in the shape of a spiral, comprising a layer of film affixed on its opposing edges to the liner.

FIG. 6 is a second exemplary embodiment of the liner of FIG. 1, illustrating a flat panel of film having pores formed there through for the diffusion/dispersal of the ozonated air, affixed along its peripheral edge to the liner, but for a non-porous passageway for receiving the ozonated air, forming a diffusion panel.

FIG. 7 is an alternative embodiment of the liner of FIG. 4, illustrating a thin film passageway formed in the shape of an “X”, comprising a layer of film affixed on its opposing edges to the liner, further including a non-porous passageway for receiving the ozonated air.

FIG. 8 is an isometric view of a litter bin receiving system for recycling used litter.

FIG. 9 is a side, cut-away view of the system of FIG. 8, wherein used litter is poured into the upper bin of the unit and the ozone generator is engaged to the unit.

FIG. 10 is a side, cut-away view of the system of FIG. 9, wherein ozonated air is pumped through porous sidewalls in the upper bin via the ozone generator, infusing the litter and waste with ozone.

FIG. 11 is side, cut-away view of the ozonated, dried litter of FIG. 10 after processing, providing recycled litter.

FIG. 12 is a side, cut-away view of the system of FIG. 11, wherein the floor of the upper bin is removed, dumping the recycled litter into a lower drawer for removal and re-use.

FIG. 13 is a side, cut-away, partially cross-sectional view of the liner of FIG. 6, illustrating the passage of the air/fluid mixture through the passageway formed between the upper film layer and the liner, and the diffusion of ozonated air through the diffusion panel into the cat litter.

FIG. 14 is a side, cut-away view of the diffusion panel of FIG. 13.

FIG. 15 is a side, isometric view of another alternative system of the present invention, wherein there is shown a hanger having a portable deodorizing fluid dispenser or generator associated therewith, here showing an ozone generator, the hanging elements of the hanger also forming conduits for the conveyance of ozone or other deodorizing fluid therethrough.

FIG. 15A is a side, close up view of the diffusers of FIG. 15.

FIG. 16 is a side, isometric view of another alternative system of the present invention, wherein there is shown a hanger having a portable deodorizing fluid dispenser or generator associated therewith, here showing an ozone generator, the diffuser formed into the liner itself via strips of film having side edges affixed to the liner, which may be apertured.

FIG. 16A is a top view of an alternative portable deodorizing fluid dispenser, wherein the diffuser is formed as a component of the unit housing.

FIG. 16B illustrates the diffuser of FIG. 16 shown as a circular apertured strip formed as a component of the liner.

FIG. 17 is a side, view of another liner embodiment of the present invention, this one in the form of a garment bag having a zipper closure or the like for enclosing clothing or the like therein, the inner wall of the liner having formed therein a length of tube forming a fluid diffuser/application area, and a length of tube forming a fluid conveyance area, for transporting fluid from the fluid dispenser or generator to the diffuser/application area.

FIG. 17A is a side view of the film tube forming the fluid conveyance area of FIG. 17.

FIG. 17B is an end view of the film tube of FIG. 17A.

FIG. 17C is a side, cut-away view of the film tube of the diffuser/application area of FIG. 17.

### DETAILED DISCUSSION OF THE INVENTION

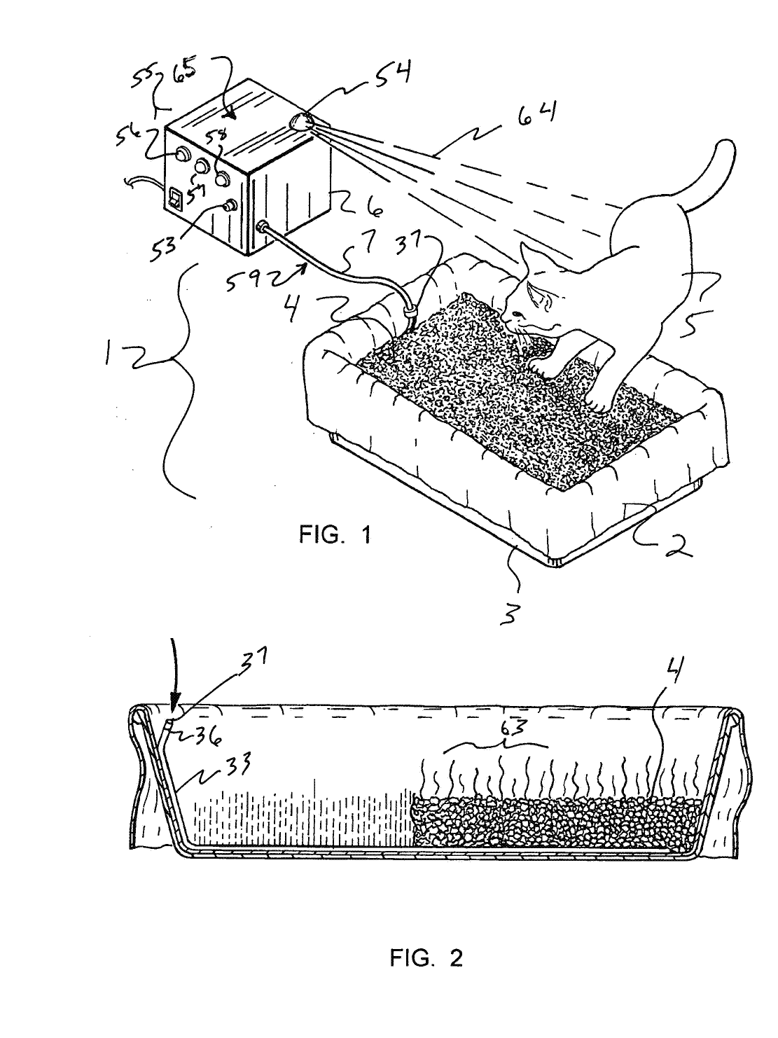

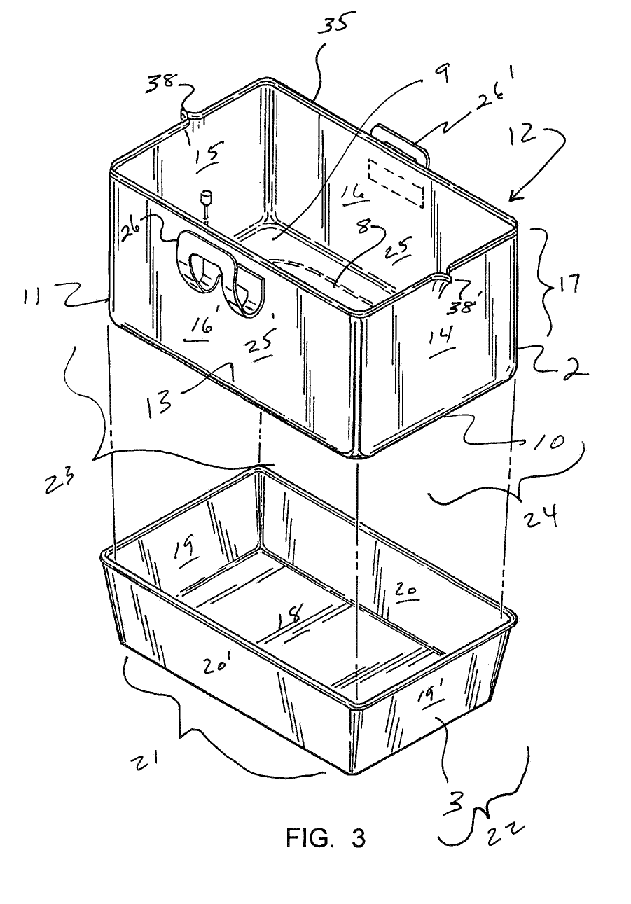

Referring to FIGS. 1-3, an exemplary embodiment of the present invention 1 comprises a liner 2 for a litter tray 3 containing litter 4 for use by an animal 5 such as a cat, for example, for the deposit of waste thereon.

A fluid deodorizer such as an ozone/air gas mixture is provided by a generator 6 which is directed via a tube 7 or the like to a diffuser 8 associated with the base 9 (the support surface upon which the litter is placed) forming the liner. The liner preferably has first 10 and second 11 ends having first 14 and second 15 side walls emanating therefrom, the liner further having first 12 and second 13 sides having first 16 and second 16′ sidewalls emanating therefrom, the sidewalls engaging the end walls to form a containment area there between.

As shown, the litter tray 3 or container has a base 18 or support surface having first 19 and second 19′ end walls and first 20 and second 20′ sidewalls which join to form a containment area, the pan having a length 21 and width 22.

The liner 2 has a length 23 and width 24 sufficient to cover and envelope the containment area of the litter tray, the side and end walls of the liner preferably having a height 17 greater than the walls of the litter tray to allow a portion of the liner to be folded over the walls of the litter tray 3 for securing and adequately lining same. An exemplary measurement of the liner may be, for example, about 11″ width by 17″ length by 4″ sidewall height.

The liner is preferably formed a flexible or flaccid film, the main portion of the liner forming inner 25 and outer 25′ walls, the inner 25 walls forming the containment area, the outer 25′ walls engaging the litter tray 3 or the like.

The liner may have mounted to its outer walls 25 in the vicinity at the sidewalls 20, 20′ first 26 and second 26′ handles, to facilitate disposal of spent litter, as will be more fully disclosed herein.

The end 19, 19′ and side 20, 20′ walls forming the containment area preferably include a top edge 35, and the top edge may be medially divided 38, 38′ to provide first 39 and second 39′ (or male/female) interlocking strips to allow the liner upper edge to be sealed to form a container for the spent waste, like a bag, with the handles 26, 26′ facilitating carrying thereof. Alternatively a draw cord may be provided along the periphery of the mouth or the bag, or a twist-tie may be used to seal the bag for containing the waste/litter for disposal or the like.

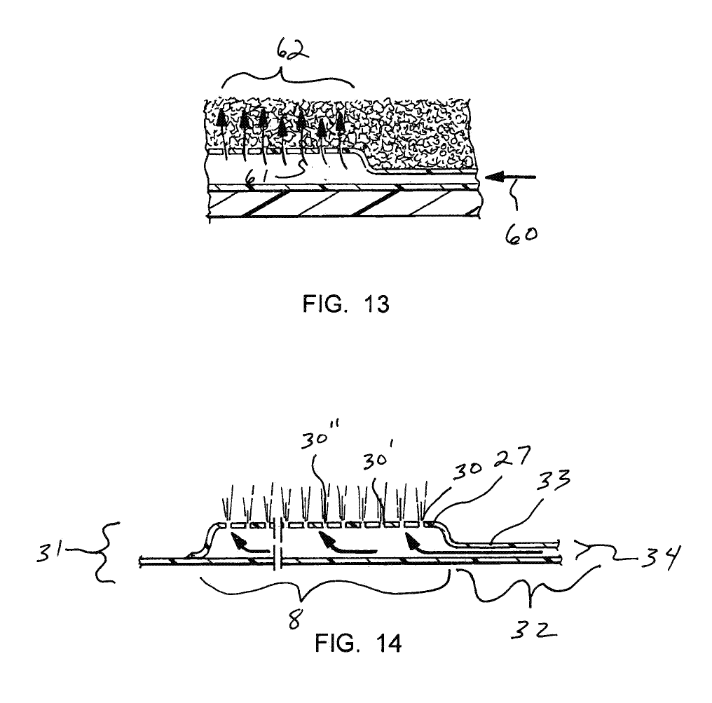

Continuing with FIGS. 1, 2, 6, 13, and 14, the base or support surface of the preferred embodiment of the liner may have associated therewith a diffuser or the like for distributing a deodorizing fluid such as an air/ozone mixture there through. The diffuser can take various configurations, and may be formed by a manifold-like tube having apertures formed along its length, or a film panel.

In the preferred, exemplary embodiment of the present invention, the diffuser 8 is formed of a film panel 27 having a periphery 28 having an edge, wherein the periphery is sealed 29 to the liner along its edge. The panel has formed there through a plurality of spaced apertures 30, 30′, 30″, the panel formed to be spaced 31 from the liner upon application of fluid (such as air/ozone mixture) there through.

The panel can be of various sizes or configurations, preferably distributing via the perforations or apertures 30 (or other fluid permeable construction) the deodorizing fluid over an area comprising the base or support surface of the liner.

A passageway 32 communicates with the diffuser panel to deliver the deodorizing fluid from the generator. The passageway can be in the form of a conventional tube, or (as shown in the referenced figures) may be in the form of a contiguous, elongated strip of film sealed 33 along its side edges to the liner, and formed to provide a space there between to form a conduit for the transport of the fluid there through the conduit having an end 37 situated distal the panel, along one of the side or end walls forming the liner. The end 37 may include a connector 37 for connecting a tube or the like thereto, so as to communicate with the generator for other deodorizing fluid source. Alternatively, a panel can be formed on the outer wall of the liner, with the apertures formed through the liner, so as to provide the deodorizing gas, through the apertures formed through the liner, and to the area to be deodorized.

As shown in FIG. 5, the diffuser may take the form of a manifold-like distribution means comprising a length of tubing having apertures (or other fluid permeable construction) formed along its length for the distribution of deodorizing fluid there through, the tubing shown in the configuration of a spiral 40 or the like. This tube may be referred to as a bubble wand or strip

As shown in FIG. 7, as an alternative to the above disclosed panel construction (which could have radial edges, linear edges, and may be rectilinear, for example), a diffuser may be formed by strips of perforated (apertures, slits, or otherwise fluid permeable) film sealed along its opposing side edges to form a permeable conduit for diffusion of various configurations, such as, for example, an “X” 41, or other configurations.

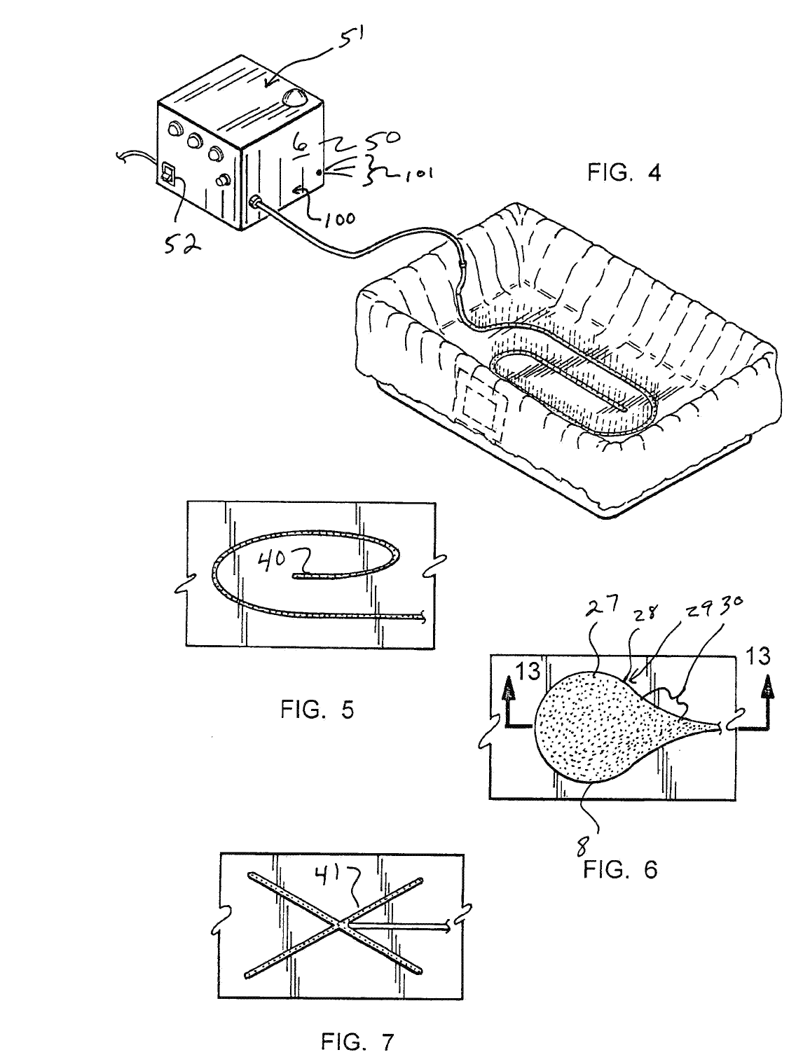

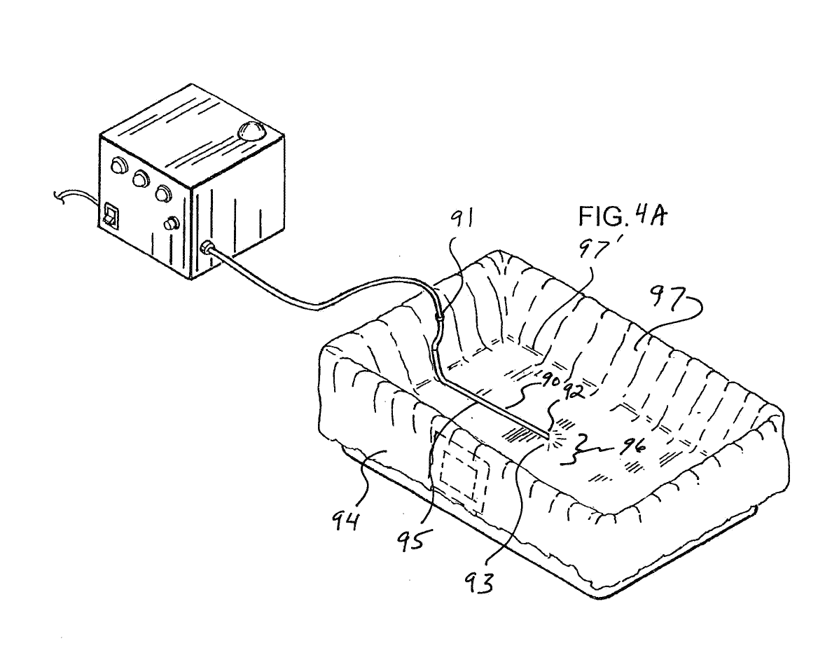

Referring to FIG. 4A, the system of the present invention may not require a diffuser, the ozone/air mixture pumped 96 directly into the litter from the open end of the hose or conduit. In use, the litter, being granular, disperses the ozone, which is heavier than air, while the liner contains the ozone, so that the litter is in effect infused in an ozone (or other deodorizing fluid) bath.

As shown, the hose 90 has first 91 and second 92 ends, the second end preferably being situated in about the lower portion 97′ of the sidewalls 97, or center 93 of the liner 94. The hose is preferably affixed 95 or formed as part of the liner, but it is not required.

Continuing with FIGS. 1, 2, 4, 13 and 14, the disinfectant fluid source can be from a variety of sources, the exemplary embodiment in the form of an generator 6 having a cabinet 50 containing an ozone generation apparatus 51, which may be controlled by a power switch 52 to initiate power to the unit, which may be powered via a wall plug, internal battery, or other power source.

The unit of the exemplary embodiment includes a proximity sensor 54, which can utilize infrared (IR) or microwave movement detection to sense the presence of an animal in the vicinity of the litter tray. Accordingly, the proximity sensor 54, in use, should be oriented to detect or sense 64 the animal 5 in the presence of the litter tray 3. Upon detection, the unit can be programmed to initiate a timer circuit 64 upon detecting the animal, the timer deferring disinfecting fluid flow until some period of time after detection has been made, for example, 5 minutes, to allow the animal to complete its business at the litter tray.

Alternatively, the unit can be programmed to initiate the flow of disinfecting fluid after detection of the animal, and after a predetermined period of the sensor sensing no activity so as to discern the animal has left the area. The unit can be further programmed such that the disinfecting flow of fluid to the diffuser is halted if during the time of flow the animal returns to the area. Upon the animal leaving the area, the flow could be made to resume.

Alternatively, the sensor can be separated from the generator 6 by wires or wireless RF communication, and can be mounted to the tray or liner, for example.

After the predetermined period of time has lapsed, the ozone generation apparatus 51 (or other disinfectant fluid) initiates and provides a fluid flow 59 (in the exemplary embodiment, an air/ozone mixture) through the tube 7, through 60 conduit 33 along the liner, to the diffuser, where it passes 61 through the perforations or apertures 30 so that it is diffused 62 through the litter 4, permeating 63 the litter and any animal waste therein. The timer circuit may be programmed to allow ozone generation/fluid flow for a pre-determined period of time, for example, 15 minutes, to deodorize, disinfect, and treat any waste left in the litter by the animal.

Ozone is heavier than air, and will fill a container in an undisturbed atmosphere like a liquid. In the present invention, the timer circuit may be programmed for a period of time sufficient to allow the ozone generator to disperse sufficient ozone/air mixture into the lined tray containing the litter/waste to fill the tray to its upper edge, in effect immersing the waste/litter in ozone, infusing same with the ozone treatment fluid.

As an example, in a conventional lined tray measuring 11″ wide by 17″ long by 6″ high, filled with 25 lbs or natural (clay) litter, an ozone generator having a flow rate of 2-3 liters per minute at 0.05-02 mPas, with ozone generation of 200 mg/hr and a with a hose situated upon the liner under the clay to disperse the ozone/air, tests indicate that it takes about four minutes for the ozone to fill the tray up to the top edges of the sidewalls and begin to spill over, in a sealed room with no air flow. Thus, the timer in this example may be programmed to run four minutes for treatment.

The ozone generator may further feature a control interface including indicator lights 55, which may include a timer circuit initiated light 57 to indicate the timer circuit has been initiated, a power light 56 to indicate power, and a cycling light 58 which indicates the disinfecting fluid flow (in the exemplary embodiment ozone/air mixture) for a predetermined period of time.

The ozone generator may further include an aerosol dispenser which may be timed to emit a spray of deodorizer into the area after the ozone has been dispensed, which can further freshen the air as well as depleting the remaining ozone in the area

A litter renewal cycle switch 53 may also be provided for use with renewing spent litter utilizing the litter renewal bin, as will be further discussed below.

It is noted that the diffuser of the present invention need not be affixed to or otherwise associated with the liner, and may comprise a structure independent of the liner, upon which could be poured the litter, or which may be otherwise buried into the litter.

It is also noted that a liner is not always required, and the diffuser of the present invention may be utilized without a liner, and that the litter tray or box may have the diffuser structure incorporated as a component therein.

What is important is that the diffuser of the present invention be associated with the litter so as to diffuse into the litter and waste therein the treatment gas, whether it be ozone or another treating or deodorizing fluid.

### Exemplary Specifications:

### Liner:

Material: High Density Polypropylene (The material indicated is for exemplary purposes. Other materials which are ozone (or other disinfecting fluid used) resistant, and which can contain the litter/waste could likewise be satisfactorily used.)
Thickness: 3 mils

### Measurements:

Size: 11″wide by 17″ long by 8″ high (sidewalls)
Diffusion Aperture size: apertures can vary significantly in size and configuration, and may alternatively take the form of slits, cuts, or porous materials. Also, a diffusion panel may not be required, a hose having an open end situated in the litter, which granular form would appear to diffuse, distribute or disperse the ozone/air to fill the tray may be adequate.

### Ozone Generator:

Type: corona field (various types may be utilized)
Flow Rate: 1-3 liters/minute (exemplary embodiment 1.5 liters/minute
Ozone concentration: 50-260 milligrams/hour

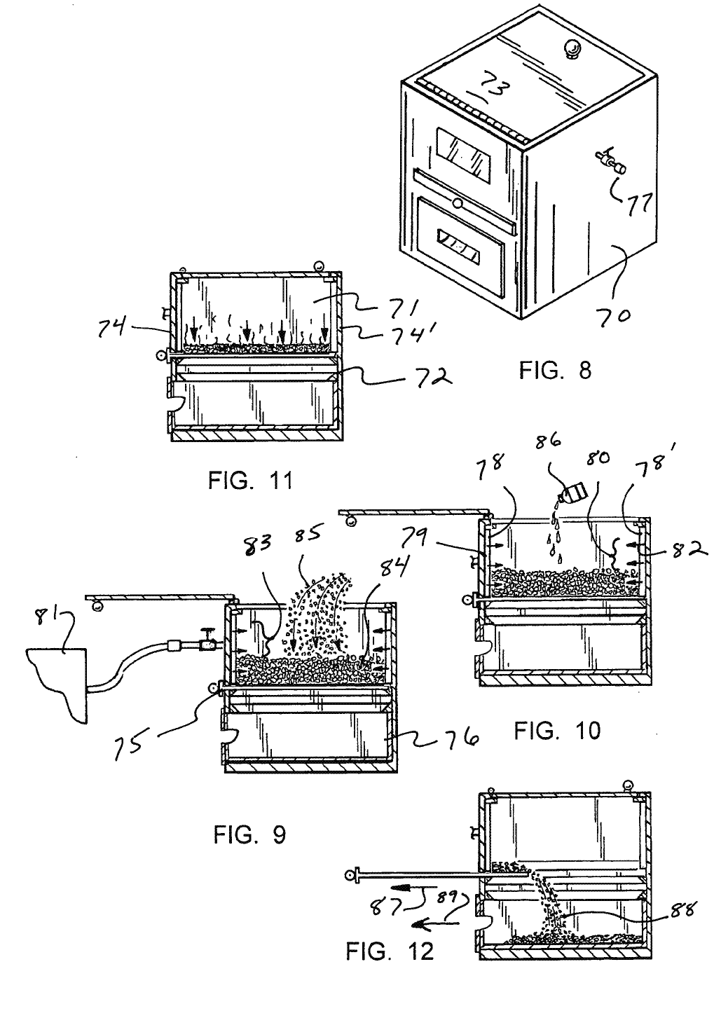

Referring to FIGS. 8-12, the present invention also contemplates a recycle bin system which may be utilized with the disinfectant fluid generator (shown as an ozone generator 51 in FIG. 4) discussed supra.

As shown, the recycle bin 70 of the present invention includes upper 71 and lower 72 compartments, the upper compartment having a lid 73 and formed by first 74 and second 74′ sidewalls, and a false (removable) floor 75 having a drawer 76 thereunder.

Situated in spaced 79 fashion from the sidewalls 74, 74′ are first 78 and second 78′ diffusion walls, respectively, each diffusion wall having formed there through a plurality of perforations or apertures for the flow of gaseous fluid there through, while being small enough to retain the litter and waste. A hose coupling is provided to communicate with a disinfecting fluid source (i.e., ozone generator), to direct the disinfecting fluid to the space 79 between the sidewalls and the diffusion walls.

In use, a litter/waste mixture 84 is poured 85 into the upper bin. Essential (scented oils) may be added to reduce the odor. The disinfecting fluid 82 is piped from the fluid source 81 via coupling 77 to the space 79 between the sidewalls and the diffusion walls, where the disinfecting fluid (i.e., ozone/air mixture) is diffused 83 via apertures 80 into the litter/waste mixture. A recycle button (53 in FIG. 1) may be initiated to start the ozone (or other disinfecting fluid generator) for the period of time required to recycle the litter/waste mixture, for example, 3 hours.

After recycling has occurred, the false floor 87 may be slidingly removed, causing the recycled litter to pour 88 into the underlying drawer 76, at which point the drawer may be removed 89 and the litter reused in a litter tray, for example.

Referring to FIG. 1, to prevent the liner from being oxidized by the ozone, the ozone generator may further include a cumulative timer which indicates when it is time to change the bag via a light or display, ex, after 50 hours of total operation. After changing the bag, the user could, for example, push a button, to reset the timer.

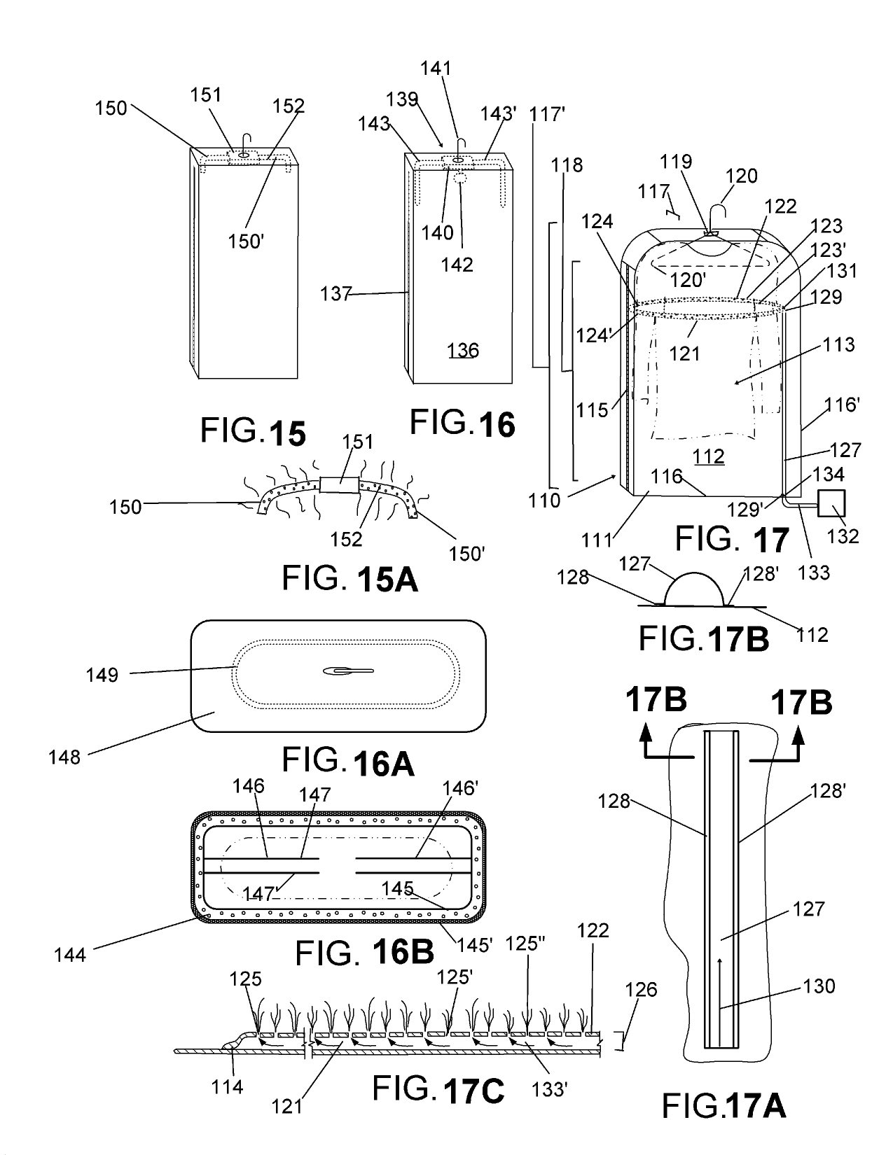

Referring to FIGS. 17, 17A and 17B, a first alternative embodiment 110 of the present invention is a system which is suitable for freshening clothing or the like, comprises a bag 111 (shown as a garment bag) forming a liner 112 to envelop an object 113 such as clothing or the like. It is noted that, while the present illustration (as well as FIGS. 15 and 16) shows the bag 111 enveloping clothing, these systems can be also used to freshen and/or deodorize or disinfect other objects such as bedding, pillows, etc; accordingly, the present description of the objects being treated is not intended to be limiting. As shown, the liner 112 has inner 116 and outer 116 walls, a width 117 and a length 117′, the inner walls forming an interior for containing the object(s) to be treated. The bag 111 forming the liner 112 has a zipper 115 or other selectively sealable feature having a length 118 sufficient to allow the selective passage of objects into the liner for treatment and or storage, and removal as desired.

As a garment bag, the bag 111 has an opening 119 for passage of the hook portion 120 of a coat hanger therethrough, with the body portion 120′ situated in the bag, for supporting clothing of the like. Alternatively, a hook can be provided emanating from the bag, with a loop within the bag for receiving hangers therein (as shown in FIG. 116).

Continuing with FIGS. 17, 17A and 17B, the inner wall 116 of the liner 112 has associated therewith a diffuser 121 or the like for distributing a deodorizing fluid such as an air/ozone mixture there through. As shown, the diffuser is situated along the inner side and end walls of the liner so as to provide deodorizing fluid about the object to be treated. If the diffuser is situated in the path of the zipper or other opening, it ideally should be sealed (see seal 114 in FIG. 17C) at those points so as to provide even dispersion of the fluid, while not interrupting the operation of the zipper or the like. The diffuser can take various configurations, and may be formed by a manifold-like tube having apertures formed along its length, or a film panel.

In the preferred, exemplary embodiment of the present invention, the diffuser 121 is formed of a first strip of film 122 having first 123 and second 123′ edges and first 124 and second 124′ ends. The strip of film 122 has formed there through a plurality of spaced apertures 125, 125′, 125″. The first 123 and second 123′ edges of the strip of film 122 affixed to the liner with the middle layer unattached along its length so that the area of the strip between the edges can be spaced 126 from the liner upon application of fluid (such as air/ozone mixture) there through. Alternatively, the strip can be formed on the outer 116′ wall of the liner 112, with the apertures formed through the liner so as to provide the deodorizing gas to the interior of the liner.

The diffuser can be of various sizes or configurations, preferably distributing via the perforations or apertures 125 (or other fluid permeable construction) the deodorizing fluid over an area comprising the interior of the liner.

A conduit 127 is formed by a contiguous, elongated strip 128 of film having first 128 and second 128′ side edges, and first 129 and second 129′ ends, the first 128 and second 128′ side edges affixed or sealed to the liner with the middle layer unattached along its length so that the area of the strip between the edges can be spaced, forming a conduit for the passage of fluid therethrough. The first end 129 of the strip 128 of film is situated to communicate 131 with the diffuser 121, the second end 129′ is situated to receive deodorizing and/or freshening fluid such as ozone (ozone/air gas mixture) from an deodorizing fluid dispenser 132, shown as an ozone generator or the like distal the diffuser, preferably via a hose or tube 133. The second end 129′ may include a connector 134 or the like affixed thereto, so as to communicate with the generator (or other fluid source) or tube leading thereto.

While a diffuser is shown, the system of the present invention may not require a diffuser, the ozone/air mixture pumped directly into the enclosed and sealed bag (garment bag) from the open end of the hose or conduit. The bag being enclosed, the ozone or other deodorizing gas will fill the space within the enclosure, permeating the object therein (in this case, clothing) and deodorizing/freshening same.

Alternatively, the unit can be programmed to initiate the flow of disinfecting fluid upon activation by the user and, after a predetermined period of time of fluid flow, the flow can be halted.

After the predetermined period of time has lapsed, the deodorizing fluid dispenser 132 (i.e., ozone generator or other disinfectant fluid source) initiates and provides a fluid flow (in the exemplary embodiment, an air/ozone mixture) through the tube 133, through 130 conduit 127 along the liner, to the diffuser, where it passes 133′ through the perforations or apertures 125, 125′, 125″ so that it is diffused through the object 113 to be deodorized (in this case, clothing), permeating same. The timer circuit may be programmed to allow ozone generation/fluid flow for a pre-determined period of time, for example, four minutes, to deodorize, disinfect, and treat the objects.

A choice of essential oils may be utilized via separate spray by the user or incorporated into the disinfecting fluid stream, to enhance the disinfecting and refreshing, but is not essential. When the bag is opened the ozone is released safely into the atmosphere along with the essential oil fragrance.

Ozone is heavier than air, and will fill a container in an undisturbed atmosphere like a liquid. In the present invention, the timer circuit may be programmed for a period of time sufficient to allow the ozone generator to disperse sufficient ozone/air mixture into the liner to fill the same, in effect immersing the contents in deodorizing gas or other fluid infusing same with the treatment fluid.

As an example, in a garment bag or liner measuring 20″ wide by 4″ deep by 28″ high, having four or five articles of adult clothing therein, an ozone generator having a flow rate of 2-3 liters per minute at 0.05-02 mPas, with ozone generation of 200 mg/hr and a with a hose engaging conduit 127 to the diffuser 121 to disperse the deodorizing gas (in this example, ozone/air), it is estimated that it would take a little over four minutes for an ozone/air mixture to fill the sealed liner/garment bag. Thus, the timer in this example may be programmed to run about four minutes for treatment.

The ozone generator may further feature a control interface including indicator lights, which may include a timer circuit initiated light to indicate the timer circuit has been initiated, a power light to indicate power, and a cycling light which indicates the disinfecting fluid flow (in the exemplary embodiment ozone/air mixture) for a predetermined period of time.

The ozone generator may further include an aerosol dispenser which may be timed to emit a spray of deodorizer (which may include the essential oils earlier referenced) into the area after the ozone has been dispensed, which can further freshen the air as well as assist to deplete the remaining ozone in the area.

Continuing with 16 and 16A, liner 136 is shown in the form of a bag having a side zipper 137, forming an enclosure for clothing or the like therein. The present embodiment differs from the above embodiment of the liner in that a portable deodorizing fluid dispenser 139 (shown in the form of a portable ozone generator) is associated with the upper portion of the liner, comprising a housing 140 for the deodorizing fluid dispenser apparatus (again, in the exemplary embodiment shown in the form of an ozone generator), the housing shown may have a hanger hook 141 emanating therefrom for hanging the bag and its contents, and on the section of the housing below the hanger hook a loop 142 for receiving hanger hooks, so as to allow coat hangers or the like to engage same for hanging clothing within the liner.

As shown, first 143 and second 143′ diffuser strips (shown in phantom in the form of apertured strips of film with edges sealed to the liner to form an apertured conduit similar in construction to diffuser 121, above) are formed to communicate via sealed connector with opposing ends of the housing, to distribute the deodorizing gas through the interior of the liner structure, to treat any contents therein.

FIG. 16B illustrates an alternative diffuser configuration to that of FIG. 16, wherein the diffuser 144 is shown as a circular apertured strip with side edges 145, 145′ sealed to the liner to form an apertured conduit therein, and first 146 and second 146′ film strip conduits, again formed by sealing the opposing side edges 147, 147′ to the liner, are provided to convey the deodorizing fluid from the deodorizing fluid dispenser to the diffuser.

As another alternative housing to the housing of FIG. 16, an alternative housing 148 can have dispensing apertures formed therethrough for directly dispensing the deodorizing fluid into the garment bag, as shown in the alternative housing view of FIG. 16A, which shows a top view of the garment bag with the diffuser aperture 149, situated below the housing distal the hanger hook, shown in phantom.

Referring to FIGS. 15 and 15A, still another alternative of the deodorizing fluid dispenser of FIG. 16 entails the use of first 150 and second 150′ diffuser support members emanating from the housing 151, said deodorizing fluid dispensers formed to support clothing as a coat hanger or the like, as an alternative to the first and second diffuser strips (143, 143′ respectively in FIG. 16. In use, the fluid dispenser would dispense deodorizing fluid (such as ozone) from the housing through the diffusion support members 150, 150′, which is diffused through the apertures 152 into the clothing or other objects to be deodorized.

It is reiterated that, although this liner is taught in conjunction with the hanging of clothing or the like, the liner is also very suitable for receiving bedding or other objects to deodorize same, and the present system is not intended to be limited as to clothing by any means.

### LISTING OF ELEMENTS

| Element | Description |
|---|---|
| 1 | System |
| 2 | liner |
| 3 | litter tray/container |
| 4 | litter |
| 5 | animal (cat) |
| 6 | ozone or other gaseous disinfectant |
| 7 | tube |
| 8 | diffuser |
| 9 | base or lower horizontal panel of liner |
| 10 | first end |
| 11 | second end |
| 12 | first side edge |
| 13 | second side edge |
| 14 | first end wall |
| 15 | second end wall |
| 16 | first, second side walls |
| 17 | side wall height |
| 18 | pan base |
| 19 | first, second pan end walls |
| 20 | first second pan side walls |
| 21 | length pan |
| 22 | width pan |
| 23 | length liner |
| 24 | width liner |
| 25 | inner, outer walls |
| 26 | first, second handles |
| 27 | diffuser as panel |
| 28 | edge about periphery |
| 29 | sealed to liner at edge |
| 30 | plurality of spaced apertures |
| 31 | panel formed to be spaced from liner upon application of |
| | fluid there through |
| 32 | passageway |
| 33 | contiguous film sealed on edge, no apertures to form conduit |
| 34 | formed to be spaced from liner for conduit |
| 35 | edge |
| 36 | end |
| 37 | connector |
| 38,′ | medially divided |
| 39,′ | zip-lock portions |
| 40 | spiral |
| 41 | “X” |
| 50 | cabinet |
| 51 | ozone generation apparatus |
| 52 | power switch |
| 53 | manual cycle switch |
| 54 | proximity sensor |
| 55 | indicator lights |
| 56 | power light |
| 57 | timer circuit initiated light |
| 58 | cycling light |
| 59 | air/ozone mixture |
| 60 | passes |
| 61 | passes |
| 62 | diffused |
| 63 | permeates |
| 64 | senses |
| 65 | timer circuit |
| 70 | recycle bin |
| 71 | upper |
| 72 | lower compartments |
| 73 | lid |
| 74,′ | sidewalls |
| 75 | false floor |
| 76 | drawer |
| 77 | hose coupling |
| 78 | diffusion wall |
| 79 | spaced |
| 80 | apertures |
| 81 | fluid source (i.e. generator) |
| 82 | disinfecting fluid |
| 83 | diffused |
| 84 | litter/waste mixture |
| 85 | poured |
| 86 | essential (scented) oils |
| 87 | false floor slidingly removed |
| 88 | recycled litter into drawer |
| 89 | removed and reused |
| 90 | hose |
| 91 | first end |
| 92 | second end |
| 93 | center |
| 94 | liner |
| 95 | affixed |
| 96 | pumped |
| 97,′ | sidewalls, lower portion |
| 100 | ozone dispenser |
| 101 | spray |
| 110 | first alternative embodiment |
| 111 | bag |
| 112 | liner |
| 113 | object |
| 115 | zipper |
| 116,′ | inner, outer walls |
| 117,′ | liner width, length |
| 118 | zipper length |
| 119 | coat hanger passage |
| 120,′ | hook portion body portion |
| 121 | diffuser |
| 122 | strip of film |
| 123.′ | first, second edges |
| 124,′ | first, second ends |
| 125,′.″ | spaced apertures |
| 126 | spaced film forming conduit |
| 127 | conduit |
| 128 | strip |
| 129,′ | first, second ends |
| 130 | through conduit |
| 131 | communicate w/diffuser 121 |
| 132 | ozone generator |
| 133 | tube |
| 134 | connector |
| 136 | liner |
| 137 | bag |
| 138 | zipper |
| 139 | deodorizing fluid dispenser (shown ozone generator) |
| 140 | housing |
| 141 | hanger hook |
| 142 | loop |
| 143,′ | first and second diffuser strips |
| 144 | diffuser |
| 145,′ | side edges |
| 146,′ | first, second film strip conduits |
| 147,′ | side edges |
| 148 | alternative housing |
| 149 | diffuser apertures |
| 150,′ | diffusion support members |
| 151 | housing |
| 152 | apertures |

The invention embodiments herein described are done so in detail for exemplary purposes only, and may be subject to many different variations in design, structure, application and operation methodology. The use of “diffuser”, “manifold”, and like terms are not intended to be limiting, referencing the dispersal, dispensing, and distribution of the fluid into the tray container area. Thus, the detailed disclosures therein should be interpreted in an illustrative, exemplary manner, and not in a limited sense.

What is claimed is:

1. The method of deodorizing an animal litter box having a bottom and side walls, comprising the steps of:
a. providing a liner of fluid impermeable, flexible film having dimensions to form a fluid impermeable layer over the walls of the animal litter box;
b. forming an elongated strip of fluid impermeable film having a length having first and second ends, and first and second edges;
c. affixing said first and second edges of said strip of film to said liner, so as to form a conduit between said first and second edges, with said strip of film forming a first wall of said conduit, and said liner forming a second wall of said conduit, said conduit having first and second ends;
d. placing said liner in said litter box;
e. covering said second end of said conduit with absorbent litter;
f. allowing an animal to deposit waste upon said absorbent litter;
g. initiating a flow of treatment fluid through said first end of said conduit;
h. allowing said treatment fluid to flow through the length of said conduit;
i. dispersing said treatment fluid into said absorbent litter via said conduit;
j. repeating steps f-i.

4. The method of claim 1, wherein after step “c” there is further provided the additional step “ci.” of forming perforations in said strip of film in the vicinity of said second end of said conduit, so as to provide a diffuser, while allowing the remainder of said conduit to remain fluid impermeable, for the passage of gas therethrough, such that fluid passing into said first end of said conduit is directed through a fluid impermeable passage formed by said conduit, to said diffuser formed in the vicinity of said second end of said conduit.

5. The method of claim 4, wherein said second end of said conduit is sealed to facilitate passage of fluid through said perforations forming said diffuser.

6. The method of claim 5, wherein said treatment fluid comprises ozone gas.

7. The method of treating clothing, comprising the steps of:
a. providing a liner of fluid impermeable, flexible film having dimensions to enclose said clothing;
b. forming an elongated strip of fluid impermeable film having a length having first and second ends, and first and second edges;
c. affixing said first and second edges along said length of said strip of film to said liner, so as to form a conduit between said first and second edges, with said strip of film forming a first wall of said conduit, and said liner forming the lower wall of said conduit, said conduit having first and second ends;
d. configuring said liner to form an enclosure for receiving said clothing;
e. sealing said liner;
f. initiating a flow of a treatment gas through said first end of said conduit;
g. allowing said treatment gas to flow through the length of said conduit;
h. dispersing said treatment gas into said clothing via said conduit;
i. repeating steps f-h.

8. The method of claim 7, wherein in step “a” said liner is formed of thermoplastic film.

9. The method of claim 8, wherein in step “b” said strip of film is formed of thermoplastic film;

10. The method of claim 9, wherein after step “c” there is further provided the additional step “ci.” of forming perforations in a strip of film in the vicinity of said second end of said conduit, so as to provide a diffuser such that said treatment gas passing into said first end of said conduit is directed through a fluid impermeable passage formed by said conduit, to said diffuser formed in the vicinity of said second end of said conduit.

11. The method of claim 9, wherein said treatment fluid comprises ozone gas.

12. The method of claim 11. wherein in step “g” said treatment gas is dispersed through said conduit in a measured volume so as to substantially fill said enclosure, so as to contact said treatment gas with the clothing therein.

10. The method of deodorizing items, comprising the steps of:
a. providing a liner of fluid impermeable, flexible film having dimensions to enclose said items;
b. forming an elongated strip of fluid impermeable film having a length having first and second ends, and first and second edges;
c. affixing said first and second edges of said strip of film to said liner, so as to form a flexible conduit between said first and second edges, with said strip of film forming a first wall of said conduit, and said liner forming a second wall of said conduit, said conduit having first and second ends;
d. placing said items within said liner;
e. sealing said liner to form a sealed enclosure having said items therein;
f. initiating a flow of treatment fluid through said first end of said conduit;
g. utilizing said conduit to contain and direct said treatment gas through the length of said conduit so as to disperse said treatment fluid into said enclosure and throughout said items;
i. repeating steps f-h.

11. The method of claim 10, wherein said items for deodorizing comprise clothing, and in step “a” there is included the additional step “a1” of forming said liner into a garment bag.

12. The method of claim 10, after step “c” there is provided the added step “c1” of forming a second elongated strip having first and second ends, and first and second edges, “c2” affixing said first and second edges of said second strip to said liner, so as to form a second conduit between said first and second edges, with said strip of film forming a first wall of said second conduit, and said liner forming said second wall of said second conduit, “c3” engaging said conduit to said second conduit so that fluid from said conduit passes into said second conduit; and forming passageways through one of said first or second walls of said second conduit, so as to form a diffuser for diffusing treatment gas into said enclosure.

13. The method of claim 10, wherein there is provided after step “e.” the added step “e1” providing a treatment fluid dispenser, said treatment fluid dispenser formed to engage said liner, said treatment fluid dispenser having a housing.

14. The method of claim 13, wherein in step “g” said treatment fluid dispenser comprises an ozone generator, and in step “g” said treatment fluid comprises ozone.

15. The method of claim 13, wherein in step “g” said treatment fluid dispenser dispenses essential oils, and in step “g” said treatment fluid comprises essential oils.

16. The method of claim 14, wherein in step “a” there is further included the step “a1” of forming said liner into a garment bag, and wherein from one end of said housing there is provided a hook protruding therefrom, said hook emanating from said garment bag, and wherein from another end of said housing there is provided a bracket for receiving hanger hooks.

17. A system for deodorizing and freshening clothing and/or bedding, comprising:
a bag forming an enclosure for clothing and/or bedding, said garment bag being sealable;
a portable fluid treatment source associated with said bag, said portable fluid treatment source having a housing, said portable fluid treatment source formed to provide to said enclosure a deodorizing fluid.

18. The system of claim 17, wherein said fluid treatment source comprises an ozone generator, and said deodorizing fluid comprises ozone.

19. The system of claim 18, wherein said bag comprises a garment bag, said housing has first and second ends, said first end having a hook emanating therefrom which is situated exterior said garment bag, said second end having a bracket emanating therefrom to engage and support clothes hangers.

20. The system of claim 19, wherein there is provided a diffusing conduit situated in said enclosure engaging said ozone generator, said diffusing conduit positioned to disperse ozone about said clothing and/or bedding, so as to deodorize and freshen same.

21. The system of claim 20, wherein said diffusing conduit comprises an elongated strip of film sealed at opposing side edges with said liner so as to form a conduit for the passage of ozone therethrough.

22. The system of claim 20, wherein said diffusing conduit is formed in the shape of a clothes hanger, to support clothing within the garment bag while allowing the diffusion of ozone therethrough.
